(19) United States
(12) Patent Application Publication
(10) Pub. No.: US 2012/0316498 A1
(43) Pub. Date: Dec. 13, 2012

(54) Intravenous fluid monitoring device

(76) Inventors: Frank Dos Santos, Suffolk, VA (US); Chi Nguyen, Suffolk, VA (US)

(21) Appl. No.: 13/489,942

(22) Filed: Jun. 6, 2012

Related U.S. Application Data
(60) Provisional application No. 61/494,951, filed on Jun. 9, 2011.

(51) Int. Cl. A61M 5/44

(52) U.S. Cl. 604/114

(57) ABSTRACT

A device for automatic monitoring intravenous fluid flow rate and temperature. The inventive device is capable of automatically adjusting the temperature of the fluid administered to a patient and recording the fluid flow rate and temperature. The device is also capable of transferring data to an electronic data storage repository via a wire connection or wirelessly.

### CROSS-REFERENCES TO RELATED APPLICATIONS

This application claims the benefit of U.S. Provisional Application No. 61/494,951, filed 9 Jun. 2011, which is incorporated herein by reference.

### BACKGROUND OF INVENTION

1. Field of Invention

The inventive subject matter relates to a device capable of monitoring multiple parameters of intravenously (IV) introduced fluids. The device is also capable of adjusting the IV fluid to a pre-set temperature level, recording measured IV parameters reporting the data through a wireless system to data monitoring stations.

2. Background Art

Intravenous (IV) administration of fluids is a commonly used method for the purpose of providing nutrition, restoring lost fluids, electrolytes, maintaining fluid balance or for administering medications directly into the circulatory system. Current methods of IV administration of fluids is primarily via a reservoir whereby the fluid is gravity fed into the IV tube. Regulation of fluid flow is often by mechanical regulation of the drip rate through a drip chamber. While regulation of the drip rate by this method is often adequate, the system requires constant monitoring in order to ensure that the IV is operating properly and that the fluid flow is at a prescribed rate. In areas where patients are receiving relatively large volumes of fluid, such as injured soldiers or accident victims, there is often a poor record of the various amounts of fluids and rates of delivery. Furthermore, the amount of fluid administered and its temperature is of considerable importance in patient outcome. In combat situations, establishing and monitoring this information in an efficient, timely and constant basis is often very challenging. Furthermore, current methods of fluid delivery do not adequately monitor fluid temperature or make temperature adjustments.

### SUMMARY OF THE INVENTION

The invention relates to small, clip-on IV monitoring device. In a preferred embodiment of the invention, the device is compatible with standard IV systems. The inventive device utilizes fittings that accept standard IV line sets with leuir lock adapters and retractable collars.

In a further embodiment, the device capable of non-intrusively monitoring fluid flow. Additionally, the device is capable of monitoring the temperature of the fluid as it flows through the device. In this aspect of the invention, the device would be capable of automatically warming the temperature the fluid to a pre-determined temperature prior to reaching the patient.

In a still further embodiment, the device would also be capable of recording the fluid monitoring data. An alternative feature of the invention is would enable the device to wirelessly download the recorded data.

### BRIEF DESCRIPTION OF THE DRAWINGS

FIG. 1. Diagram of operation of the inventive device illustrating basic aspects.

FIG. 2. Illustration of disposable warming cartridge showing heating tube and connection points to the IV tube.

FIG. 3. Schematic showing cross-section of warming cartridge.

FIG. 4. Illustration showing the disposable warming cartridge and the physical association of it with the control unit.

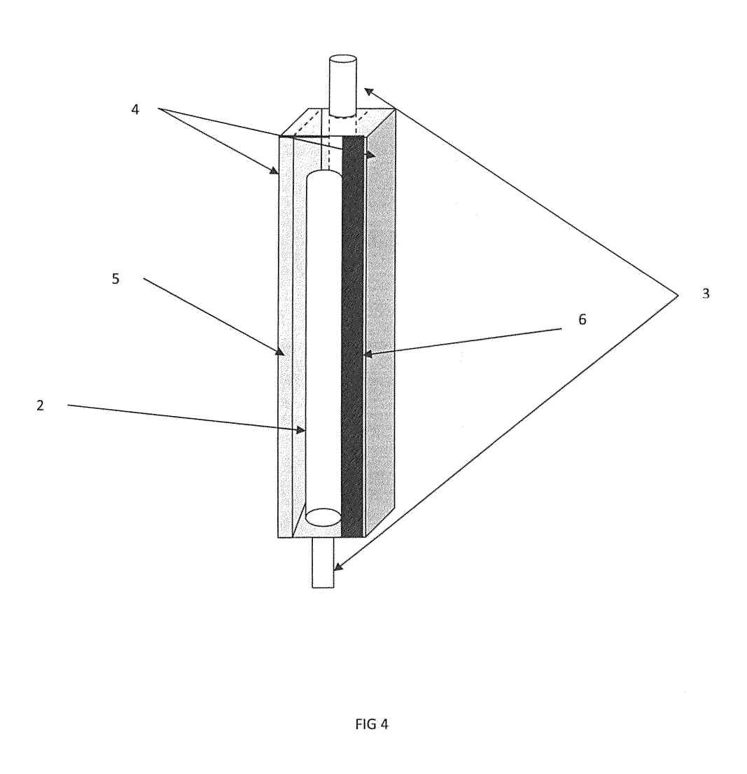

### DETAILED DESCRIPTION OF PREFERRED EMBODIMENTS

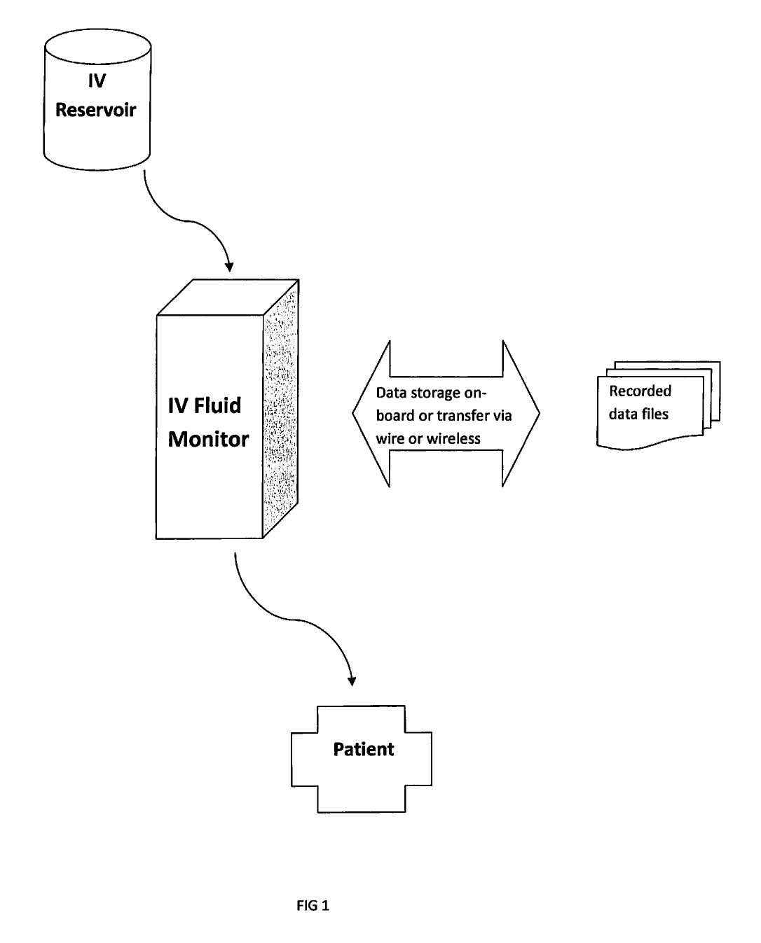

The invention relates to a small, clip-on intravenous (IV) fluid monitoring device for intravenous fluid administration. The device is capable monitoring and recording relevant IV delivery parameters and adjusting IV fluid temperature. FIG. 1 illustrates a summary of the operational aspects of the inventive device.

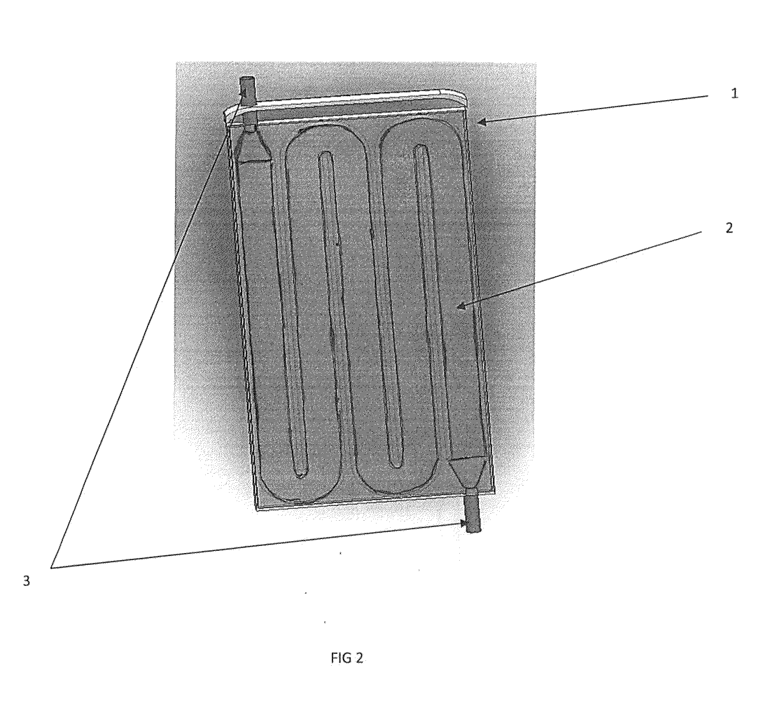

In a preferred embodiment, the device is compatible with standard IV systems. Standard tubing from a fluid reservoir attaches to the device through any of a number of means, such as, for example, through a Leur Lock™ (Becton Dickenson, Franklin Lake, N.J.) or similar system, via connections (3) on a disposable warming cartridge (1), as illustrated schematically in FIG. 2. The warming cartridge contains a cartridge tube (2) that connects directly with the IV tubing. The warming cartridge, illustrated in FIG. 2, is disposable and attaches to a control unit (4).

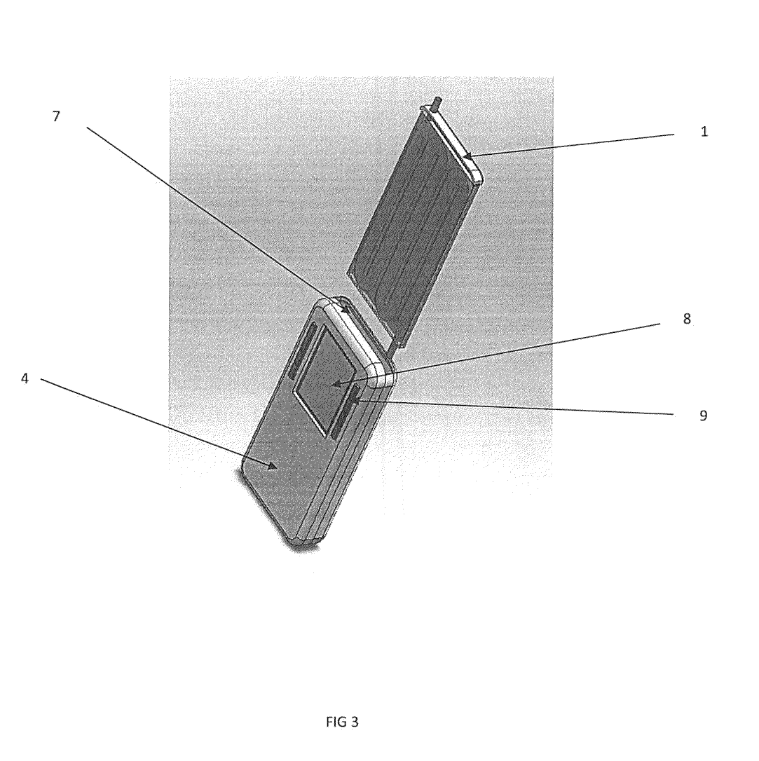

As illustrated in FIG. 3, the warming cartridge (1), is operably connected to the control unit (4). The control unit (4) contains microprocessor circuitry and programming to enable it to receive sensor input, calculate and store data during the operation of the device. In a preferred embodiment, the control unit is enclosed in an impact resistant casing and contains LCD/LED (8) display for easy monitoring of IV operations.

The control unit (4) is capable of monitoring fluid parameters, including temperature, flow volume rate and cumulative fluid volume delivered to a patient. In a further embodiment, the device is capable of non-intrusively monitoring fluid flow in order to minimize disruption of the continuity of fluid flow. In the preferred embodiment, the device would be able to measure fluid and temperature within the warming cartridge, to within +/−0.1° C. Additionally, the device would be capable of automatically warming the temperature of the fluid to 38° C. within +/−0.5° C., or other pre-determined temperature, with an input range of 20° to 38° C., prior leaving the device and reaching the patient.

In a still further embodiment, the device would also be capable of recording the fluid monitoring data. Data received from the device would be stored, via a wire or wireless connection, to a data storage device, such as a lap-top computer or other electronic data repository.

### EXAMPLE 1

### Measurement of Flow Rate

In a preferred embodiment, measurement by means that does not impede or disturb the continuity of the flow through the IV tube is preferred. In this embodiment, therefore, flow rate will be accomplished by laser velocimetry or by measurement of mass flow rate.

As an example, flow rate can be measurement by thermal mass, as molecules of a moving fluid come into contact with a heat source, they absorb heat with concomitant cooling of the heat source. At increased flow rates, more molecules come into contact with the heat source, absorbing even more heat. The amount of heat dissipated from the heat source in this manner is proportional to the number of molecules of a particular liquid, the thermal characteristics of the liquid, and its flow characteristics.

Relating heat dissipation and flow rate, the rate of heat absorbed by a flow stream is directly proportional to its mass flow according to the equation q_{w}=C·h(T_{wi}−T_{wo}), where h=the convective heat transfer coefficient and where T_{wi} and T_{wo} refer to the outside temperature of the tubing at two locations (T_{wo1} and T_{wo2}), accomplished by separate temperature sensor readings. From the convective heat transfer coefficient h (and an experimental correction factor), the wall temperature inside the tube can be obtained, i.e., T_{wi1} and T_{wi2}.

The temperature gradient value (δT_{wi}/δx) can be approximated, which correlates to the inlet Reynolds number (Re). Re is a dimensionless number that gives a ratio of inertial to viscous forces, and therefore, quantifies the relative importance of the two types of forces for the flow conditions that would be encountered in the IV tube. Laminar flow occurs at low Re, where viscous forces prevail, and is characterized by smooth, constant fluid motion. Turbulent flow occurs at high Re numbers and is dominated by inertial forces, which tend to produce eddies, vortices and other fluid flow instabilities.

With incrementation of the inlet Re number, the temperature gradient value will decrease because of the better convective heat transfer performance provided by the higher flow. Therefore, the relationship between δT_{wi}/δx and Re can be pre-measured and a relationship defined as Re=fcn·(δT_{wi}/δx). Using \dot{m}=pν(πD²)/4=Re·4/μπD, where \dot{m} is mass flow rate, the mass flow rate can be determined.

In another embodiment, fluid flow can be measured by laser velocimetry. In this embodiment, laser Doppler velocimetry is used to cross two beans of collimated, monochromatic and coherent laser light in the flow stream of the cartridge tubing (2) of the warming cartridge (1). The beams can be generated by separate sources or by splitting a single beam. The two beams are then aimed to intersect at the focal point of a laser beam (waists), either in the warming cartridge (1) or within the controlling unit (4), where they interfere and generate a set of straight fringes. The sensor is then aligned to flow such that the fringes are perpendicular to the flow direction. As particles pass through the fringes, they reflect light into a photodetector in the control unit (4). By measuring the Doppler frequence-shift of the scattered light, the velocity and hence the flow of the liquid, is measured. In an alternative embodiment, fluid flow is measured by a Doppler vibrometer. In this embodiment, a beam of monochromatic laser light is sent into the flow, and particles are caused to reflect light creating a Doppler shift. The shift corresponds to the particle velocity, which translates into fluid flow rate.

In a preferred embodiment, the control unit (4) programming would calculate the fluid temperature inside the IV tube, and consequently calculate the flow rate by thermal mass. The flow rate and temperature can be set so that adjustments to flow rate can be automatically adjusted by the inventive device to a pre-set value.

### EXAMPLE 2

### Warming of Fluid

Similarly, the device will be capable of warming the IV fluid automatically to a pre-set value from 20° to 38° C. +/−0.5° C. Warming of IV fluid is accomplished within the cartridge tubing (2) contained and integral with the warming cartridge (1). The warming system uses resistive heating material that is controlled to precisely apply the heat as the fluid flows through the disposable cartridge (1).

In one embodiment, the warming cartridge tubing (2) is composed of thermally conductive metal-alloy tubing. In this embodiment, the surfaces of the cartridge (1) that contact the tubing are made of similar metal alloys as the cartridge tubing (2) such as the heating surface (5).

The association of heating surface (5), cartridge tubing (2) and thermal insulation (6) is illustrated in FIG. 3. Direct contact of the heating surface (5) of the warming cartridge (1) to the integral cartridge tubing (2) reduces heat loss associated with metal to plastic connection. Opposite to the heating surface (5) is a thermal insulation (6) to further reduce heat loss from the device and increase the efficiency of heat transfer to the IV fluid.

Logic controlled inductive heating is implemented to reduce the risk of overheating and compromising the integrity of fluid. The amount of heat applied to the fluid is determined by the fluid flow rate, which will be independently measured, as illustrated in the embodiments described in Example 1.

### EXAMPLE 3

### Storage of IV Fluid Data

In a preferred embodiment, the control unit (4) contains an embedded microprocessor that controls functions of the device, including heating of IV fluid, in response to pre-set temperatures and measured temperature of the IV fluid moving through the unit. In the embodiment, the control unit (4) would contain microprocessor circuitry to enable the control unit (4) to electronically and automatically record the flow rate measurements. Additionally, in another embodiment, the control unit can monitor aggregate fluid volume and the data stored.

Data received from the device would be stored, via a wire connection, to a data storage device, such as a lap-top computer or other electronic data repository. Alternatively, data would be transferred to an electronic data repository via a wireless connection, such as via Bluetooth®, Kirkland, Wash. (USA). In a preferred embodiment, data from the control unit would be “time-stamped” and be Erasable Programmable Read-Only Member (EEPROM). This would ensure that data is not alterable and would enable the data to be safely saved upon removal of power to the device. In a wire or wireless mode, the data would be transferred from the control unit (4) either at preset times or on command. It is contemplated that control of the operation of the device via the control unit (4) can be achieved either through a control panel (9), FIG. 3, on the control unit (4) or by remote control.

In a preferred embodiment, the device can be operated external from a dwelling or hospital power source. This will enable the device to be utilized in operation settings where electrical power outlets are not available. Considering the power requirement to operate the heating surface (5) as well as the microprocessor unit and associated sensors, approximately 21 Watt-hours is required in order to raise a 1 liter bag of 0.9% saline from 18° C., which is slightly below room temperature, to 38° C. Since a typical, required flow rate will be approximately 150 mL/min, the heat transfer is estimated to require approximately 188 Watts. Therefore, based on these likely parameters, in a preferred embodiment the power source will be supplied by a lithium-ion battery. In this embodiment, the microprocessor can be provided operating power for up to 6 hours of continuous operation.

The embodiment can also incorporate a modular battery pack that, in combination with EEPROM, enables battery packs to be exchanged in order to extend operation of the device without interruption of service. The batteries can be either recharged using an external battery charger or by using a universal serial bus (USB) connector that is built into the device. The USB connection can also be utilized to power the device when hospital or building electrical power (e.g., 110 volt-AC) is available. The USB also permits secondary wired communications capability with USB enabled devices.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

What is claimed is:

1. An intravenous (IV) fluid monitoring device comprising:
a warming cartridge comprising an internal resistive heating surface, an internal insulting surface and a cartridge tube, wherein said cartridge tube passes between said heating surface and insulating surface and is in contact with said heating surface; multiple thermal microsensors sensors at the inflow and outflow ends of the cartridge tube, for measuring temperature on the outside surface of said cartridge tube; a control unit connected to said warming cartridge, containing microprocessor circuitry and programming to enable it to receive sensor input, wherein said control unit is capable of monitoring the temperature and flow rate of fluid through the cartridge tube and adjust the temperature of fluid flowing through the cartridge tube, by controlling the resistive heating surface, according to a pre-set temperature.

2. The intravenous fluid monitoring device of claim 1, wherein said control unit monitors flow rate and determines the flow rate of fluid passing through the cartridge tube using input from the multiple thermal microsensors.

3. The intravenous fluid monitoring device of claim 1, wherein said thermal microsensors are also located mid-flow, between the inflow and outflow ends.

4. The intravenous fluid monitoring device of claim 1, wherein said monitoring of temperature and flow rate of fluid is non-intrusive.

5. The intravenous fluid monitoring device of claim 1, wherein said control unit can transmit the flow rate and temperature of input flow and output flow by wire or wireless means to a data storage device.

6. The intravenous fluid monitoring device of claim 1, wherein said warming cartridge and cartridge tube are composed of metal-alloy.

7. The intravenous fluid monitoring device of claim 1, wherein said warming cartridge can warm fluid flowing through it to 38° C. within +/−0.5° C.

8. The intravenous fluid monitoring device of claim 1, wherein said flow rate is measured by thermal mass.

9. The intravenous fluid monitoring device of claim 1, wherein said flow rate is measured by laser velocimetry.

10. The intravenous fluid monitoring device of claim 1, wherein said means for attaching to an intravenous tubing is a clip.

11. The intravenous fluid monitoring device of claim 1, wherein said warming cartridge is disposable.

12. The intravenous fluid monitoring device of claim 1, wherein said control unit is controlled via a control panel or by wire or wireless remote control.

13. The intravenous fluid monitoring device of claim 1, wherein said control unit contains an impact resistant outer casing.

14. The intravenous fluid monitoring device of claim 1, wherein said device is capable of battery operation for up to 6 hours.

15. The intravenous fluid monitoring device of claim 1, wherein said control unit contains a USB connection.

16. The intravenous fluid monitoring device of claim 1, wherein said control unit can monitor and stores aggregate fluid volume.
